(19) United States
(12) Patent Application Publication
(10) Pub. No.: US 2006/0258596 A1
(43) Pub. Date: Nov. 16, 2006

(54) Antimicrobial agents

(76) Inventors: Timothy Rutland Walsh, Clifton (GB); Robin Anthony Howe, Westbury-on-Trym (GB)

Correspondence Address: JENNIFER M MCCALLUM, PH D, ESQ;THE MCCALLUM LAW FIRM, LLC, 685 BRIGGS STREET, PO BOX 929, ERIE, CO, 80516, US

(21) Appl. No.: 10/545,962

(22) PCT Filed: Feb. 14, 2004
(86) PCT No.: PCT/GB04/00592

§ 371 Date: Feb. 10, 2006

(30) Foreign Application Priority Data
Feb. 14, 2003 (GB) 0303375.0
Feb. 14, 2003 (GB) 0303369.3

(51) Int. Cl. C07K 7/06; A61K 38/08; C07H 21/04; C12P 21/06; C12N 1/21

(52) U.S. Cl. 514/16; 530/329; 435/69.1; 435/252.35; 435/471; 536/23.7

(57) ABSTRACT

An antibacterial peptide obtainable from a S. mitis and S. oralis; or a variant thereof, or a fragment of any of these, for use as an antibacterial agent. Specific peptides have been identified. These peptides are secreted by strains of S. mitis or S. oralis. They are active against inter alia gram negative bacteria and Staphylococcal spp. The peptides may be isolated for use or the S. mitis or S. oralis may be used in a probiotic therapy.

The present invention relates to antimicrobial agents, in particular peptides which are active inter alia against gram-negative bacteria, and which are obtainable from strains of Streptococcus mitis or Streptococcus oralis.

Bacteriocins are one of a number of antimicrobial substances produced by lactic acid bacteria (LAB), including organic acids, hydrogen peroxide, diacetyl and inhibitory enzymes. They have been more strictly defined as “proteinaceous compounds which kill closely related bacteria”; however, some are known to have broad-range inhibitory qualities. Their antimicrobial action is almost exclusively bacteriocidal (Mc Auliffe et al., 2001 FEMS Microbiology Letters. 25, 285-308; Van Kraaij et al., 1998, Biochemistry. 37, 16033-16040).

Molecules include non-bacterial products like cecropin (produced by insects), indolicidin (bovine neutrophils), ranalexin, magainin and buforin (bullfrogs) as well as bacterial products such as macedoin (Streptococcus macedonicus), SalA (Streptococcus salivarius), bovicin (Streptococcus bovis), pediocin (Pediococcus spp.), mutacin (Streptococcus mutans) and the well studied nisin (Lactococcus lactis) (Hillman, J. D., 2002. Genetically modified Streptococcus mutans for the prevention of dental caries. Antoine van Leeuwevhoek. 82, 361-366; Venema, K. 1995. Trends in Microbiology. 3, 299-304, Montville, T. J. et al., 1998. Applied Microbiological Biotechnology. 50, 511-519).

Various groups have defined a number of distinct of bacteriocins; Class I are small (<5 kDa) peptides containing the unusual amino acids lanthionine and β-methyl-lanthionine e.g. nisin. Class II bacteriocins are small (5<kDa), heat stable, non-Lan containing membrane active peptides e.g. pediocin. Members of Class III are large (<30 kDa), heat labile proteins. A fourth class has been suggested which include non-proteinaceous moieties for activity.

Whilst these some of these molecules have been studied for their antimicrobial activity, in the main, these studies have been limited to inhibiting bacteria responsible for food spoilage such as Listeria monocytogenes. However, two studies have been undertaken with respect to studying their effect on Gram-negative bacteria. The first of these examined nisin and non-bacterial peptides alone and in combination with clinically used antibiotics against Pseudomonas aeruginosa (Giacometti, A et al., 1999, Journal of Antimicrobial Chemotherapy. 44, 614-645). In this study the activity of nisin was unremarkable exhibiting a minimal bacteriocidal concentration of 16->128 mg/l. The second study investigated the antimicrobial effects of the non-bacterial peptides buforin, cecropin and magainin and whilst some activity could be demonstrated, no bacterial peptides were included in this study (Giacometti, A et al., 2000, Antimicrobial Agents and Chemotherapy. 44, 1716-1719).

Gram-negative bacteria can be broadly grouped into two groups:
1. Fermentors such as Escherichia coli and Klebsiella pneumoniae; and
2. Non-fermentors such as Pseudomonas aeruginosa, Acinetobacter spp. and Stenotrophomonas maltophilia.

The first group are derived from normal gut flora and can cause a range of infections including urinary tract infections. The second group are organisms chiefly found in the environment such as soil and water sources and can cause severe infections in the immunocompromised, burns patients and cystic fibrosis patients. These bacteria are also of particular concern to intensive care units. Despite the fact that the “fermentors” are becoming increasingly resistant to standard antimicrobial therapy such as ampicillin and ciprofloxicin, drugs such as ceftazidime and imipenem, for the most part, still guarantee cover.

However, the non-fermentors have at their disposal an array of antibiotic resistant mechanisms that when combined means that infection caused by them are virtually untreatable. These bacteria are also receptive to DNA plasmids encoding additional enzymes, such as metallo-β-lactamases, which are capable of hydrolysing all classes of β-lactams including the carbapenems, usually the last drug of choice to eradicate such bacteria. Thus, conventional antibiotics are not successful in the treatment of infections by these bacteria.

It is important to note that in the last five years there have been many new anti-Gram positive drugs such as synercid, daptomycin, linezolid, oritavancin and anti-MRSA β-lactams. However, there appears to be a lack of molecules active against gram-negative bacteria, either which have been released or which have been announced as entering phase I clinical trials. There appears then to be a window of at least five years in which treatment of infection by highly resistant non-fermenting bacteria will be difficult. During that time period, the resistance of the bacteria to conventional antibiotics is likely to increase further.

It has been reported previously (Cystic Fibrosis Symposium, Stockholm, 2000) that the normal flora (in this case from cystic fibrosis patients) has an antimicrobial effect on Gram-negative bacteria.

The group of Streptococci known as viridans Streptococci or “Streptococcus viridans” make up a portion of the normal flora of the nasopharynx and are considered harmless until they get into sterile sites such as the blood where they can cause infective endocarditis.

Streptococcus salivarius is one member of the group of viridans Streptoccocci. This strain, (Ross, K. F. et al., 1993. Applied Environmental Microbiology. 59, 2014-2021) as well as other members of this group such as Streptococcus mutans (Hillman, 2002 supra.) have been known to produce bacteriocins such as mutacin and SalA that have been shown to have antimicrobial action against other “normal mouth flora”. These bacteriocins are generally proteins of high molecular weight.

The applicants have identified a new antimicrobial agent that is effective against bacteria and in particular Gram-negative bacteria.

According to the present invention, there is provided an antibacterial peptide obtainable from Streptococcus mitis or

Streptococcus oralis; or a variant thereof, or a fragment of any of these. The peptide is isolated or purified. Furthermore, it is suitable for use in therapy.

As used herein, the term “peptide” refers to short sequences of of amino acids, in particular of less than 20 amino acids, suitably less than 15 amino acids in length, more suitably less than 12 amino acids in length, preferably less than 10 amino acids in length. Amino acids contained within the peptide may be modified, for example by dehydration, phosphorylation or glycosylation. In particular, any serine or tyrosine residues may be dehydrated.

Preferably, the peptides are obtainable from Streptococcus mitis.

As used herein, the expression “variant” refers to sequences of amino acids, which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

Broadly speaking, fewer non-conservative substitutions will be possible without altering the biological activity of the polypeptide. Suitably variants will be at least 60% identical, more suitably at least 70% identical, yet more suitably at least 80%, preferably at least 90% and possibly at least 95% identical to the base sequence.

Identity in this instance can be judged for example using the BLAST algorithm or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The expression “fragment” as used herein refers to any portion of the given amino acid sequence that has antibacterial activity. Fragments will suitably comprise at least 5, for instance from the basic sequence. More than one such fragment may be joined together.

Suitably the peptide of the invention has a molecular weight of less than 2,300 Da, preferably less than 1,000 Da, for instance, less than 800.

In one embodiment, the antibacterial peptide is a peptide obtainable from Streptococcus mitis, or a variant thereof, or a fragment of any of these.

Streptococcus mitis also belongs to the group of viridans Streptococcus. It is described by Barsotti et al. (2002) Research Microbiology 153:687091. Strains constituting S. mitis can be identified by amplifying the hyper variable region of the 23s-rDNA and sequencing the amplicons, as described for example by Rudney J. D et al, Oral Microbiology and Immunology (1999) 14, 33-42.

S. mitis has not previously been identified as a source of small antibiotic peptides, and is a particularly preferred source of peptides of the invention.

Preferably the antibacterial peptide of the invention is a peptide obtainable from Streptococcus mitis or a variant thereof.

The applicants have found that peptides comprising a chain of largely hydrophobic amino acids can have antibacterial activity.

A particular example of such a peptide is a peptide comprising at least seven amino acids of SEQ ID NO 1:

| | |
|---|---|
| X¹X⁷X²X³X⁴X⁵X⁶ | (SEQ ID NO 1) |

where X²is an amino acid with an uncharged polar side chain;

X³is a tyrosine, threonine or serine; and

and X¹, X⁴ and X⁶ are uncharged non-polar amino acids,

and X⁵ is a charged amino acid

and X⁷ is cysteine or histidine.

Amino acids with an uncharged polar side chain include serine, tyrosine, threonine asparagine,and glutamine.

Uncharged non-polar amino acids include glycine, alanine, valine, leucine, isoleucine, proline, phenylalanine, methionine, tryptophan or cysteine.

Examples of charged amino acids include lysine, arginine, histidine, aspartic acid or glutamic acid.

X² is suitably serine.

Preferably X³ selected from tyrosine, serine or threonine and preferably it is tyrosine.

X¹, X⁴ and X⁶ are suitably independently selected from isoleucine, leucine, alanine or valine.

In particular X¹ is leucine.

Preferably X⁹ is isoleucine.

Preferably X⁶ is valine.

X⁵ is suitably selected from aspartic acid or glutamic acid, and is preferably aspartic acid.

X⁷ is preferably cysteine.

Thus particular examples of SEQ ID NO 1 are SEQ ID NO 2

| | |
|---|---|
| LCSYIDV | (SEQ ID NO 2) |

This peptide is a peptide known to be produce by S. mitis, but it has previously been of an unknown function. It is believed that the cysteine present in SEQ ID NO 1 forms a bridge with another amino acid in the peptide, in particular X³, which is suitably tyrosine.

Novel antibacterial peptides of SEQ ID NO 1, other than SEQ ID NO 2, form a particular aspect of the invention.

In a further embodiment, the peptide of the invention further comprises further amino acids fused to SEQ ID NO 1, at the N— and/or C-terminus. These may be derived from the protein from which the peptide is derived, or they may comprise a synthetic sequence, such as a sequence that enhances the solubility or assists with the purification or isolation of peptide.

Suitable sequences that enhance solubility of the peptide will be sequences of charged amino acids as set out above.

Examples of sequences that may assist in purification or isolation of the peptide include known tag sequences such as His tag sequences (5 or more histidine residues) and the like.

In another embodiment, the present invention provides an antibacterial peptide, which is either a peptide obtainable from Streptococcus oralis, or a variant thereof, or a fragment of any of these. Preferably the peptide is a peptide obtainable from Streptococcus oralis.

Strains constituting S. oralis can also be identified by amplifying the hyper variable region of the 23s-rDNA and sequencing the amplicons, as described for example by Rudney J. D et al, Oral Microbiology and Immunology (1999) 14, 33-42.

Peptides of the invention may be isolated from the appropriate strain of S. mitis or S. oralis using conventional methods. The peptide is a secreted peptide and therefore it may be isolated from the supernatant of a culture of the S. mitis or S. oralis.

Thus, for example, a strain of S. mitis or S. oralis Streptococci may be cultured in conventional conditions, for example at 37° C. in the presence of a culture medium. After a suitable incubation period, for example of from 12-48 hours, samples of the culture supernatant may be removed and desired proteins separated.

For instance, the supernatant may be treated with a commercial protease blocker and sodium azide (0.2%) to prevent any deterioration of the target molecules. At this stage, the proteins may be concentrated by various methods including ammonium sulphate precipitation, or ultracentrifugation or by using commercially available centricons. All these procedures are well known in the art. The use of ultracentrifugation centricons may be preferred concentration steps as neither of these options interfere with the properties of the peptides or proteins, which are kept in their native state.

Once the proteins and peptides are concentrated, for example to a concentration of from 200-400 ng/ml, mass spectral analysis can be carried out, and the antibacterial peptides identified.

Antibacterial peptides of the invention may then be identified (the antibacterial peptide obtainable from S. mitis is heat liable and sensitive to proteinase K) and isolated or purified.

Methods of obtaining antimicrobial peptides by isolating them from cultured strains of S. mitis or S. oralis form a further aspect of the invention.

Once purified, the sequence of the antimicrobial peptide from S. mitis or S. oralis may be readily determined using conventional methods.

Thereafter, peptides of the invention may be prepared using chemical methods, for example using a peptide synthesiser. Alternatively, they may be prepared using recombinant DNA methods. Broadly speaking, nucleic acids encoding the peptides of the invention are incorporated into expression vectors or plasmids using conventional methods. They may then be used to transform a host cell, which may be a prokaryotic or eukaryotic cell, but is preferably one of the known prokaryotic expression hosts, such as lactococcus, wherein that the cell is not highly susceptible to the effect of the peptide. Peptides of the invention may then be recovered from the culture.

Nucleic acids encoding the peptides of the invention, together with vectors or plasmids containing these and recombinant cells transformed with these vectors or plasmids form further aspects of the invention.

The applicants have taken bronchial lavages from cystic fibrosis patients, and found that the antimicrobial effect exhibited by the normal flora is reproducible. Isolation and characterisation of individual species within the lavages was undertaken and it was found that S. mitis, S. oralis was responsible for some of the observed antimicrobial effects.

The applicants have also found that this effect can occur against all non-fermenting Gram-negative bacteria and also many Gram-negative fermentors. In addition, antibacterial effects against Staphylococcus aureus have been found.

The antibacterial effect produced by certain peptides of the invention is one of cidality and not merely inhibition, consistent with other work on bacteriocins. This is particularly advantageous in the context of therapeutic applications. Thus in a particular embodiment, the invention provides peptides wherein the antibacterial effect is a bactericidal effect.

Thus the peptides of the invention can have a broad spectrum activity. In particular, they can be active against Enterobacteriacae (such as E. coli), Burkholderia spp., Stenotrophomonas maltophilia, and P. aeruginosa, Acinetobacter spp. and Staphylococcal infections.

For instance the applicants have found that the peptide of SEQ ID NO 2 inhibits the growth of Enterobacteriacae, Burkholderia spp., Stenotrophomonas maltophilia, and P. aeruginosa. This peptide exhibits potent antimicrobial activity, inhibiting P. aeruginosa at sub-μm and E. coli at approx. 1-5 μm (approx 1-5 ng/ml) concentrations. Moreover, our analysis shows that the peptide is bacteriocidal with 99.9% killing at its MIC concentration.

Furthermore, since this compound is produced by normal flora, it is believed to have very low toxicity. In addition, the immunogenicity of the peptide is likely to be low.

The peptides of the invention are therefore of use in therapy, in particular for the treatment of bacterial infections, for instance for the treatment of infections by Gram-negative bacteria or of Staphylcoccus aureus infections.

The peptides are suitably administered to a patient in need thereof in the form of a pharmaceutical composition, in which they are combined with pharmaceutically acceptable carriers or exipients. Such compositions form a further aspect of the invention.

Suitable carriers may be solid or liquid carriers as are known in the art.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing.

Compositions may comprise other well-known formulation additives such as one or more colouring, sweetening, flavouring, preservative agents, inert diluents, granulating and disintegrating agents, binding agents, lubricating agents, and anti-oxidants. The selection will depend upon the particular form the composition will take, and will be determined by a formulation chemist using the principles set out for example in Chapter 25.2 in Volume 5 of Comprehensive Medicinal Chemistry (Corwin Hansch; Chairman of Editorial Board), Pergamon Press 1990.

The amount of active ingredient that is combined with one or more carriers to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic purposes of the peptides of the invention will vary according to the nature and severity of the conditions, the age and sex of the animal or patient and the route of administration, and will be determined by a clinician in accordance with normal clinical practice. Generally however an antibacterial peptide as described above will generally be administered so that a daily dose in the range, for example, 0.5 mg to 75 mg per kg body weight is received.

Alternatively, nucleic acids encoding the peptides of the invention may be administered to a patient in need thereof in such way that the peptides are expressed in vivo. For instance, nucleic acids encoding the peptides may be used to transform suitable vectors such as viral or bacterial vectors, or plasmids, which may then be administered to a patient in need thereof.

In some instances, it may be convenient to administer S. mitis or S. oralis itself as the therapeutic agent. WO 99/53932 and WO90/09186 suggest that certain particular strains can be used to treat specific conditions. Strains such as S. mitis and S. oralis are in essence, commensal bacteria, and therefore should not produce any significant adverse effects in the patient, they are suitable for such therapies.

Alternatively recombinant plasmids carrying the gene encoding a peptide of the invention expressed from a donor organism such as Lactococcus could be administered as a therapeutic agent. Thus, a suitable microorganism, preferably a commensal microorganism which is not adversely affected by the peptides of the invention, such as Lactococcus is engineered using conventional DNA technology, to express the peptide of the invention, and then utilised as a therapeutic agent.

These probiotic therapies can be carried out either alone or in combination with conventional antimicrobial therapies.

The strains used will suitably be combined with a pharmaceutically acceptable carrier to form a pharmaceutical composition for administration purposes.

Thus according to a further aspect, the invention provides the use of an isolated strain of S. mitis or S. oralis or a bacterial strain engineered to express a peptide of the invention, in the preparation of a medicament for the treatment of bacterial infections selected from in particular gram negative bacterial infections, such as Enterobacteriacae, Burkholderia spp., Stenotrophomonas maltophilia, and P. aeruginosa, Acinetobacter spp. and Staphylococcal infections.

Yet a further aspect of the invention comprises a pharmaceutical composition comprising a strain of S. mitis or S. oralis.

In yet a further aspect, the invention provides a method of treating a bacterial infection, which method comprises administering to a patient in need thereof, an antibacterially effective amount of a peptide as described above.

In particular, the bacterial infections will be those caused by gram-negative bacteria, or Staphylococcus aureus. Compositions and methods for administration of the compounds, including the use of a strain of S. mitis or S. oralis, are as set out above.

The invention will now be particularly described by way of example with reference to the accompanying drawings in which:

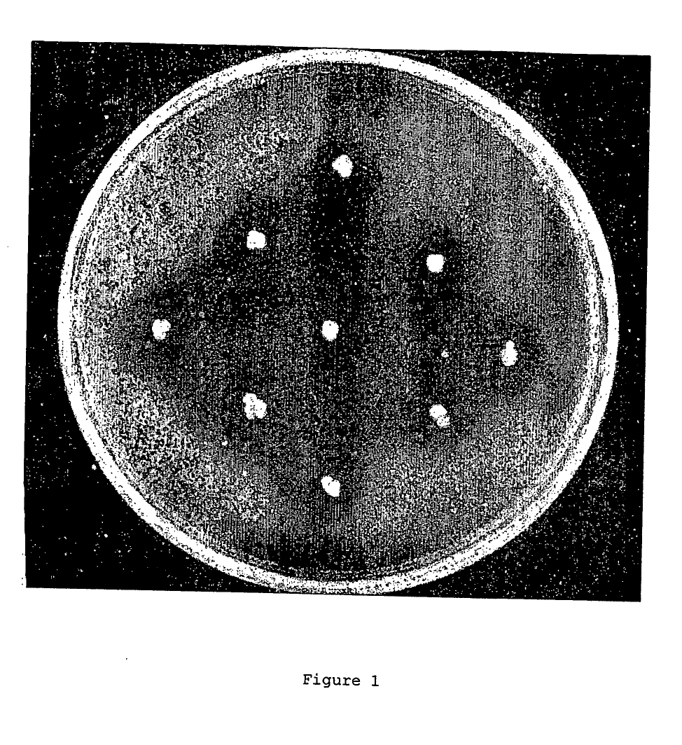

FIG. 1 shows a culture plate in which a strain of Pseudomonas aeruginosa is grown in the presence of nine strains of S. mitis;

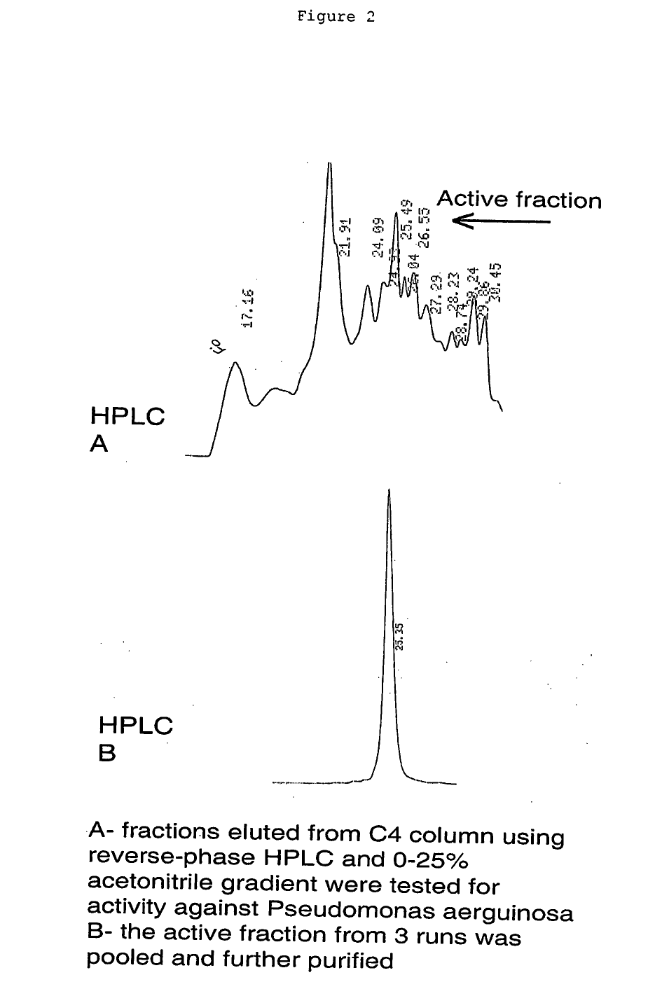

FIG. 2 shows the results of purification by HPLC of extracellular peptides secreted from S. mitis, where A shows fractions eluted from the C4 column using reverse-phase HPLC and 0-25% acetonitrile gradient were tested for activity against Pseudomonas aeruginosa, and B shows how the active fraction from 3 runs was pooled and further purified;

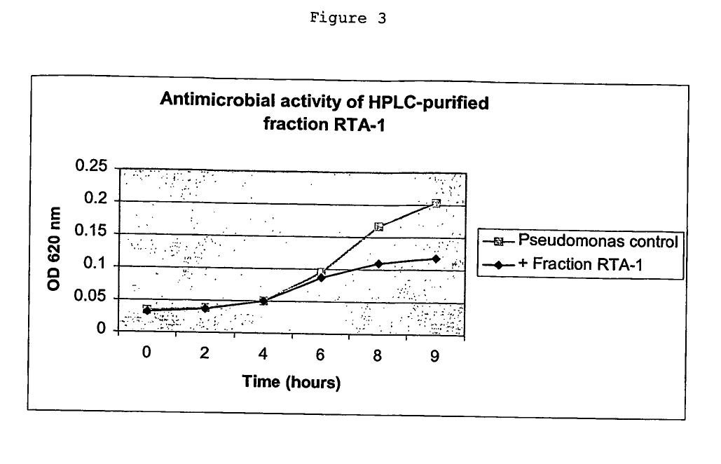

FIG. 3 is a graph showing antimicrobial activity of HPLC purified fraction of an antibacterial peptide of the invention; and

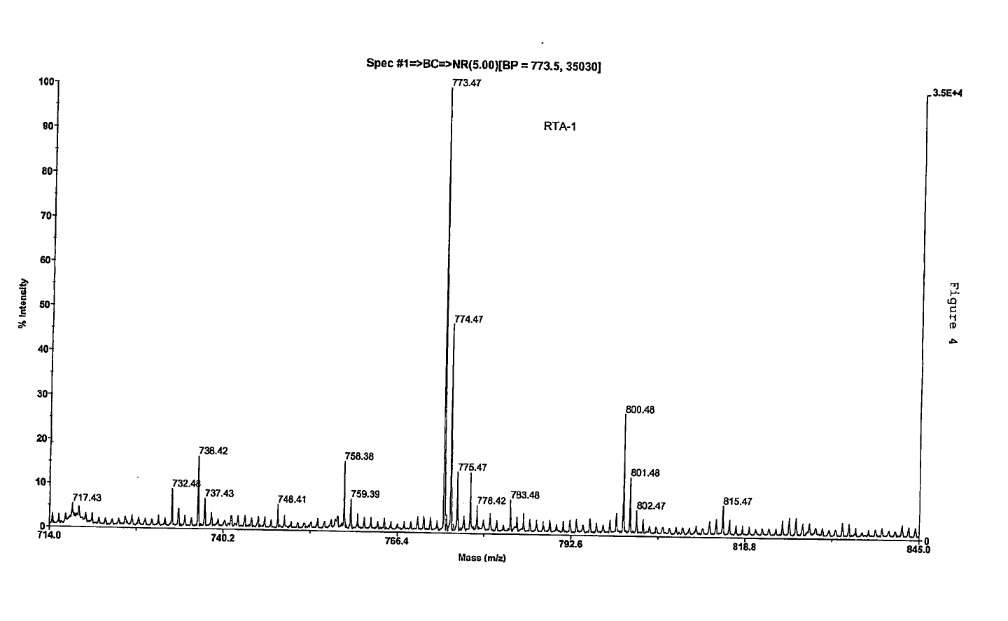

FIG. 4 illustrates the MALDI TOF mass spectrum of the peptide (RTA-1) with antimicrobial activity against P. aeruginosa. An experimental Mw 773.5 Da shown above respective peak was obtained.

### EXAMPLE 1

Nine strains of S. mitis were isolated from the normal flora of bronchial lavages. The hypervariable region of the 23s-rDNA was amplified and the amplicons sequenced to confirm their identity as S. mitis in accordance with Rudney et al. supra.

A suspension of a strain of gram-negative Pseudomonas aeruginosa, which had been isolated from a blood culture, in water, was added to an agar plate. Each of the isolated S. mitis strains were spotted onto the plate using a toothpick. The plate was cultured for 24 hours at 37° C. The resultant plate is illustrated in FIG. 1.

In this figure the light spots represent the S. mitis cultures. The darkened area shows the area of inhibition of the P. aeruginosa.

Samples of each of the S. mitis strains were then cultured overnight in a 10 ml broth. Supernatant from the culture strains was removed and added to a container of P. aeruginosa in broth. Within a few minutes, the turbidity of the culture reduced, indicating that the supernatant was having a lytic effect on the P. aeruginosa cultures. The culture was also plated onto agar, but no viable P. aeruginosa cultures appeared.

This is indicative of the presence of an antibacterially effective peptide, secreted by the S. mitis cultures. The peptide appears to have a cidal rather then merely an inhibitory effect.

### EXAMPLE 2

A strain of S. oralis was isolated from the normal flora of bronchial lavages. The hypervariable region of the 23s-rDNA was amplified and the amplicons sequenced to confirm the identity as S. oralis in accordance with Rudney et al. supra.

A suspension of a strain of gram-negative Pseudomonas aeruginosa, which had been isolated from a blood culture, in water, was added to an agar plate. The isolated S. oralis strain was spotted onto the plate using a toothpick. The plate was cultured for 24 hours at 37° C. It was clear that growth of P. aeruginosa was inhibited in the region of the S. oralis strain, indicating that the latter was secreting an antibacterial peptide.

### EXAMPLE 3

Preliminary Characterization of the antibacterial peptide from S. mitis was carried out as follows. Extra-cellularly secreted proteins from S. mitis were purified by a combination of ion-exchange, size exclusion and hydrophobic interaction chromatography.

Final purification of extra-cellular secreted peptides from S. mitis was achieved by reverse-phase HPLC using a C4 column and with a linear gradient of 0-25% acetonitrile (FIG. 2A). Of the resulting 13 fractions only the sixth (elution time of 25.49 in the figure) inhibited the growth of P. aeruginosa at the concentration tested. This fraction from three similar runs was pooled, and further purified by HPLC (FIG. 2B). The purified peptide, (named RTA-1), seems to be a constitutive secreted component of S. mitis as it is produced in the absence of environmental stimuli such as the presence of extracts of P. aeruginosa in the media.

Matrix-assisted laser desorption time of flight (MALDI-TOF) analysis of the isolated peptide gave a molecular mass of 773.5 daltons (FIG. 4)

N-terminal sequencing of this peptide revealed the sequence LCSYIDV. As the theoretical molecular mass of this sequence is 793 it is possible that the peptide is modified (dehydration of serine, or tyrosine residue) as is the case with the lantibiotics class of peptides. This sequence of RTA-1 matches a small peptide sequence of unknown origin from the S. mitis genome.

### EXAMPLE 4

Antimicrobial assays were used to confirm the activity of fractions isolated from FPLC and HPLC columns.

HPLC-purified peptide preparations were assayed for inhibitory activity against P. aeruginosa. Stock 10 nM peptide preparations were prepared in distilled water using lyophilised, HPLC-purified material. P. aeruginosa was grown to mid-logarithmic phase in Luria-Bertani (LB) medium. Bacterial culture was diluted 1:100 and 50 μl incubated in a 96-well microtitreplate with either 50 μl water (control) or 20 μl peptide+30 μl water. Bacterial growth was monitored at 620 nm.

Bacteriocidal activity against P. aeruginosa was demonstrated by removing an aliquot of the P. aeruginosa diluted in micro titre wells either with the peptide or water (control) after 9 hours incubation at 37° C., diluting 1:10000 μl, plating onto LB agar plates, and counting the number of colonies observed after 16 hours incubation at 37° C.

Typical results of the inhibition are as shown in FIG. 3. This suggests that the isolated peptide, RTA-1, displays antibacterial activity towards P. aeruginosa in concentrations that are in the order of 1 nM.

1-34. (canceled)

35. An isolated peptide selected from the group consisting of an antibacterial peptide from a strain of Streptococcus mitis, an antibacterial peptide from a strain of Streptococcus oralis, a variant thereof, and a fragment of any of these.

36. The antibacterial peptide according to claim 35 wherein the strain is Streptococcus mitis.

37. The peptide according to claim 35 wherein said peptide has a molecular weight of less than 2,300 Da.

38. The peptide according to claim 37 wherein said peptide has a molecular weight of less than 1000.

39. The peptide according to claim 38 which has a molecular weight of less than 800.

40. The peptide according to claim 35 wherein the strain is Streptococcus oralis.

41. A pharmaceutical composition comprising an isolated peptide of claim 35 and a pharmaceutically acceptable carrier.

42. A method of treating a bacterial infection in a patient comprising:
administering to the patient an isolated peptide selected from the group consisting of an antibacterial peptide from a strain of Streptococcus mitis, an antibacterial peptide from a strain of Streptococcus oralis, a variant thereof, and a fragment of any of these.

43. The method of claim 42, wherein the bacterial infection is caused by gram-negative bacteria.

44. The method of claim 42, wherein the bacterial infection is caused by Staphylococcus spp.

45. The method of claim 42, wherein the bacterial infection is caused by Enterobacteriacae, Burkholderia spp., Stenotrophomonas maltophilia, and P. aeruginosa, Acinetobacter spp. or Staphylococcal infections.

46. A method of obtaining an antimicrobial peptide according to claim 35 which method comprises culturing a strain of S. mitis or S. oralis and isolating the peptide therefrom.

47. A nucleic acid that encodes a peptide selected from the group consisting of an antibacterial peptide from a strain of Streptococcus mitis, an antibacterial peptide from a strain of Streptococcus oralis, a variant thereof, and a fragment of any of these.

48. A vector or plasmid which comprises a nucleic acid according to claim 47.

49. A recombinant cell which has been transformed with a vector or plasmid according to claim 48.


SEQUENCE LISTING (2 sequences)

SEQ ID NO: 1 - protein, 7 aa, Streptococcus mitis
  MISC_FEATURE (1)..(1): uncharged non-polar amino acid
XXXXXXX

SEQ ID NO: 2 - protein, 7 aa, Streptococcus mitis
LCSYIDV